SEQUENCE LISTING (30 sequences)

SEQ ID NO: 1 - DNA, 24 bp, synthetic construct
  misc_feature 1..24: note = primer
  source 1..24: mol_type = other DNA
ccacttccagccgaggtccttgcg

SEQ ID NO: 2 - DNA, 20 bp, synthetic construct
  misc_feature 1..20: note = primer
  source 1..20: mol_type = other DNA
gctccacggagaagaactgc

SEQ ID NO: 3 - DNA, 20 bp, synthetic construct
  misc_feature 1..20: note = primer
  source 1..20: mol_type = other DNA
gttggcatggtagcccttgg

SEQ ID NO: 4 - DNA, 24 bp, synthetic construct
  misc_feature 1..24: note = primer
  source 1..24: mol_type = other DNA
ctgctgttgctgctgccctggaca

SEQ ID NO: 5 - DNA, 20 bp, synthetic construct
  misc_feature 1..20: note = primer
  source 1..20: mol_type = other DNA
gctggggagcagagctgtaa

SEQ ID NO: 6 - DNA, 21 bp, synthetic construct
  misc_feature 1..21: note = primer
  source 1..21: mol_type = other DNA
cttctgtgaaagctgctggca

SEQ ID NO: 7 - DNA, 24 bp, synthetic construct
  misc_feature 1..24: note = primer
  source 1..24: mol_type = other DNA
accacaagcatgagccaccacgcc

SEQ ID NO: 8 - DNA, 23 bp, synthetic construct
  misc_feature 1..23: note = primer
  source 1..23: mol_type = other DNA
agcctgggaaacataacaagacc

SEQ ID NO: 9 - DNA, 21 bp, synthetic construct
  misc_feature 1..21: note = primer
  source 1..21: mol_type = other DNA
tcctgcctcatcctcctgaac

SEQ ID NO: 10 - DNA, 24 bp, synthetic construct
  misc_feature 1..24: note = primer
  source 1..24: mol_type = other DNA
ctgctcctggtgttgcctgctgcc

SEQ ID NO: 11 - DNA, 21 bp, synthetic construct
  misc_feature 1..21: note = primer
  source 1..21: mol_type = other DNA
cttcggtccagttgccttctc

SEQ ID NO: 12 - DNA, 21 bp, synthetic construct
  misc_feature 1..21: note = primer
  source 1..21: mol_type = other DNA
aagaggtgagtggctgtctgt

SEQ ID NO: 13 - DNA, 24 bp, synthetic construct
  misc_feature 1..24: note = primer
  source 1..24: mol_type = other DNA
acaccaatgcccaactgcctgcct

SEQ ID NO: 14 - DNA, 24 bp, synthetic construct
  misc_feature 1..24: note = primer
  source 1..24: mol_type = other DNA
tatagcctggactttcctgttgtc

SEQ ID NO: 15 - DNA, 20 bp, synthetic construct
  misc_feature 1..20: note = primer
  source 1..20: mol_type = other DNA
gctgactgtcctggctgatg

SEQ ID NO: 16 - DNA, 23 bp, synthetic construct
  misc_feature 1..23: note = primer
  source 1..23: mol_type = other DNA
acctgcttgtcagccagctccgg

SEQ ID NO: 17 - DNA, 21 bp, synthetic construct
  misc_feature 1..21: note = primer
  source 1..21: mol_type = other DNA
cttccttgcaggactcaccac

SEQ ID NO: 18 - DNA, 22 bp, synthetic construct
  misc_feature 1..22: note = primer
  source 1..22: mol_type = other DNA
gctgatgtgaaggtgcaaactc

SEQ ID NO: 19 - DNA, 24 bp, synthetic construct
  misc_feature 1..24: note = primer
  source 1..24: mol_type = other DNA
cggctacagcttcaccaccacggc

SEQ ID NO: 20 - DNA, 24 bp, synthetic construct
  misc_feature 1..24: note = primer
  source 1..24: mol_type = other DNA
gactacctcatgaagatcctcacc

SEQ ID NO: 21 - DNA, 23 bp, synthetic construct
  misc_feature 1..23: note = primer
  source 1..23: mol_type = other DNA
tctccttaatgtcacgcacgatt

SEQ ID NO: 22 - DNA, 24 bp, synthetic construct
  misc_feature 1..24: note = primer
  source 1..24: mol_type = other DNA
tctcagggtcctcattgcggctgc

SEQ ID NO: 23 - DNA, 24 bp, synthetic construct
  misc_feature 1..24: note = primer
  source 1..24: mol_type = other DNA
aacttgcctctcttcatgtattcc

SEQ ID NO: 24 - DNA, 21 bp, synthetic construct
  misc_feature 1..21: note = primer
  source 1..21: mol_type = other DNA
actttgcctcccagatcacag

SEQ ID NO: 25 - DNA, 20 bp, synthetic construct
  misc_feature 1..20: note = primer
  source 1..20: mol_type = other DNA
ttgatgggaagtatgccagc

SEQ ID NO: 26 - DNA, 29 bp, synthetic construct
  misc_feature 1..29: note = primer
  source 1..29: mol_type = other DNA
cggtgcctttgactctcagaacaacaccg

SEQ ID NO: 27 - DNA, 24 bp, synthetic construct
  misc_feature 1..24: note = primer
  source 1..24: mol_type = other DNA
tctttccaaattcaaacacaaagc

SEQ ID NO: 28 - DNA, 24 bp, synthetic construct
  misc_feature 1..24: note = primer
  source 1..24: mol_type = other DNA
tccgccgtccctgcttggtgatga

SEQ ID NO: 29 - DNA, 24 bp, synthetic construct
  misc_feature 1..24: note = primer
  source 1..24: mol_type = other DNA
ccaagtttaatcagcaccagacag

SEQ ID NO: 30 - DNA, 23 bp, synthetic construct
  misc_feature 1..23: note = primer
  source 1..23: mol_type = other DNA
gccacagtaaatgacaggaatgg

(19) United States
(12) Patent Application Publication
(10) Pub. No.: US 2025/0376514 A1
(43) Pub. Date: Dec. 11, 2025

(54) SIGNATURE OF TL1A (TNFSF15) SIGNALING PATHWAY

(71) Applicant: Cedars-Sinai Medical Center, Los Angeles, CA (US)

(72) Inventors: Kathrin S. Michelsen, Los Angeles, CA (US); Stephan R. Targan, Santa Monica, CA (US)

(21) Appl. No.: 19/067,260

(22) Filed: Feb. 28, 2025

Related U.S. Application Data
(62) Division of application No. 17/701,372, filed on Mar. 22, 2022, now Pat. No. 12,269,873.
Application No. 17/701,372 is a division of application No. 16/388,101, filed on Apr. 18, 2019, now Pat. No. 11,312,768.
Application No. 16/388,101 is a continuation of application No. 14/900,024, filed on Dec. 18, 2015, now Pat. No. 10,316,083.
(60) Provisional application No. 61/856,491, filed on Jul. 19, 2013.

(51) Int. Cl. C07K 16/24; A61K 39/00; A61K 39/395; A61K 45/06; C12Q 1/68; C12Q 1/6883; G01N 15/149; G01N 33/483; G01N 33/50; G01N 33/53; G01N 33/569; G01N 33/68

(52) U.S. Cl. CPC C07K 16/241; A61K 39/3955; A61K 45/06; C07K 16/244; C12Q 1/68; C12Q 1/6883; G01N 33/483; G01N 33/50; G01N 33/53; G01N 33/68; G01N 33/6863; A61K 2039/505; C07K 2317/76; C12Q 2600/106; C12Q 2600/156; C12Q 2600/158; G01N 15/149; G01N 33/56966; G01N 2333/5425; G01N 2333/70575; G01N 2800/065; G01N 2800/12; G01N 2800/24; G01N 2800/7095

(57) ABSTRACT

The present invention relates to the finding that TL1A enhances differentiation of TH17 cells, and enhance IL-17 secretion from TH17 cells. In one embodiment, the present invention provides a method of treating an inflammatory disease comprising determining the presence of a TL1A signaling profile, and treating the disease by administering a composition comprising a therapeutically effective dosage of one or more inhibitors of TL1A or TH17 cell differentiation. In another embodiment, the disease is characterized by TH17 differentiation.

### CROSS REFERENCE TO RELATED APPLICATIONS

This application is a divisional of U.S. application Ser. No. 17/701,372 filed Mar. 22, 2022, which is a divisional application of U.S. application Ser. No. 16/388,101 filed Apr. 18, 2019, now issued as U.S. Pat. No. 11,312,768 on Apr. 26, 2022, which is a continuation application of U.S. Ser. No. 14/900,024 filed Dec. 18, 2015, now issued as U.S. Pat. No. 10,316,083 on Jun. 11, 2019, which is a U.S. National Phase of International Application No. PCT/US2014/047326filed Jul. 18, 2014, which claims the benefit of U.S. Provisional Application No. 61/856,491 filed Jul. 19, 2013, the contents of each thereof are hereby incorporated by reference in their entirety.

### STATEMENT REGARDING FEDERALLY SPONSORED RESEARCH OR DEVELOPMENT

This invention was made with government support under Grant No. DK071176 awarded by the National Institutes of Health. The government has certain rights in the invention.

### SEQUENCE LISTING

The instant application contains a Sequence Listing which has been submitted in XML format and is hereby incorporated by reference in its entirety. Said XML copy, created on Feb. 27, 2025, is named 56884-722.403_SL.xml and is 38,477 bytes in size.

### BACKGROUND

All publications herein are incorporated by reference to the same extent as if each individual publication or patent application was specifically and individually indicated to be incorporated by reference. The following description includes information that may be useful in understanding the present invention. Itis not an admission that any of the information provided herein is prior art or relevant to the presently claimed invention, or that any publication specifically or implicitly referenced is prior art.

TL1A is a member of the TNF superfamily, with expression of TL1A mainly confined to inflamed tissues of colon and small bowel, and is particularly expressed on gut-homing CD4+CCR9+ mucosal T cells. TL1A has been demonstrated to synergize with IL-12 and IL-18 in the stimulation of IFN-γ by peripheral CD4+ T cells and to increase IL-2 driven proliferation and IFN-γ production by peripheral T cells. TL1A mediates a strong co-stimulation of TH1 cells by enhancing IFN-gamma production by peripheral CD4+ and mucosal CCR9+ T cells. TL1A plays an important role in the development of chronic colitis, experimental autoimmune encephalomyelitis, and allergic lung inflammation by modulating TH1, TH17, and TH2 responses suggesting an important role in chronic inflammatory processes. However, the exact mechanism how TL1A enhances TH1 and TH17 responses remains to be elucidated.

### BRIEF SUMMARY OF THE INVENTION

Various embodiments include a method of treating a disease, comprising obtaining a sample from a subject, assaying the sample to diagnose a disease based on the presence of TH17 cell differentiation, treating the subject by administering a therapeutically effective dosage of a composition comprising one or more inhibitors of TL1A. In another embodiment, the TH17 cell differentiation is from human naïve CD4+ T cells. In another embodiment, the disease is psoriasis, arthritis, experimental autoimmune encephalomyelitis, or colitis. In another embodiment, diagnosing the disease comprises determining the presence of a TL1A signaling profile. In another embodiment, the TL1A signaling profile comprises the presence of a downregulation of one or more genes listed in FIGS. 26A-26C herein. In another embodiment, the TL1A signaling profile comprises the presence of an upregulation of one or more genes listed in FIGS. 26D-26F herein. In another embodiment, the one or more inhibitors of TL1A is an inhibitor of the DR3 receptor. In another embodiment, the disease is intestinal inflammation.

Other embodiments include a method of treating an inflammatory disease in a subject, comprising determining the presence of a TL1A signaling profile in a subject, and treating the inflammatory disease by inhibiting TH17 differentiation from CD4+ T cells. In another embodiment, the TL1A signaling profile comprises the presence of a downregulation of one or more genes listed in FIGS. 26A-26C herein. In another embodiment, the TL1A signaling profile comprises the presence of an upregulation of one or more genes listed in FIGS. 26D-26F herein. In another embodiment, inhibiting TH17 differentiation comprises administering one or more inhibitors of TL1A. In another embodiment, inhibiting TH17 differentiation further comprises inhibiting IL-17 secretion from TH17 cells.

Other embodiments include a method of diagnosing a disease in a subject, comprising: obtaining a sample from the subject, subjecting the sample to an assay adapted to determine the presence or absence of elevated TL1A expression and/or one or more TL1A genetic risk variants, and diagnosing the disease based on the presence of elevated TL1A expression and/or one or more TL1A genetic risk variants in the subject. In another embodiment, the subject is human. In another embodiment, the subject is Asian. In another embodiment, the subject has one or more Th17 signature cytokines elevated. In another embodiment, the sample is taken from the peripheral blood and/or intestinal biopsy of the subject. In another embodiment, the Th17 signature cytokines include IL-17, IL-22 and/or IL-9. In another embodiment, the disease is an inflammatory disease. In another embodiment, the disease is inflammatory bowel disease (IBD). In another embodiment, the disease is rheumatoid arthritis. In another embodiment, further comprising determining the presence of a TL1A signaling profile. In another embodiment, the TL1A signaling profile comprises TL1A stimulation resulting in a downregulation of one or more genes listed in FIGS. 26A-26C herein, and/or an upregulation of one or more genes listed in FIGS. 26D-26F herein.

Various embodiments include a method of treating an inflammatory condition in a subject, comprising determining the presence of elevated TL1A expression, Th17 signature cytokines, and/or one or more TL1A genetic risk variants in the subject, and treating the inflammatory condition in the subject. In another embodiment, the subject is human. In another embodiment, the subject is Asian. In another embodiment, the inflammatory condition is inflammatory bowel disease. In another embodiment, the inflammatory condition is treated by administering a therapeutically effective dosage of inhibitor of TL1A and/or inhibitor of one or more Th17 signature cytokines. In another embodiment, the one or more Th17 signature cytokines include IL-17, IL-22 and/or IL-9. In another embodiment, the treatment is administered in conjunction with an anti-inflammatory therapeutic. In another embodiment, the treatment is administered by direct injection to the subject.

Other embodiments include a composition comprising a therapeutically effective dosage of inhibitor of TL1A and/or inhibitor of Th17 signature cytokines, and an acceptable carrier. In another embodiment, the inhibitor of TL1A is a TL1A antibody.

Various embodiments include a method of treating a disease in a subject, comprising providing a composition comprising a therapeutically effective dosage of inhibitor of TL1A and/or inhibitor of Th17 signature cytokines, and administering the composition to the subject. In another embodiment, the subject is human. In another embodiment, the disease is Inflammatory Bowel Disease (IBD). In another embodiment, the disease is arthritis. In another embodiment, the composition is administered in conjunction with treatment for inflammation.

Other features and advantages of the invention will become apparent from the following detailed description, taken in conjunction with the accompanying drawings, which illustrate, by way of example, various embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A-1G depict, in accordance with embodiments herein, FcγR stimulation in monocytes induces concomitant expression of TL1A and TGF-β1. CD14+ monocytes were stimulated with Immune Complexes (IC), LPS, Pam3, IFN-γ, Flagellin, or anti-CD40 Ab for the indicated time points. Expression of various cytokines was analyzed by real-time PCR and normalized to β-Actin expression levels. FIG. 1A shows TL1A and FIG. 1B shows TGF-β1 expression in monocytes after 16 h stimulation. FIG. 1C shows a time-course of TGF-β1 expression in monocytes stimulated with the indicated ligands. FIGS. 1D-1G show time-course of cytokine expression in monocytes stimulated with IC. One out of four representative experiments is shown.

FIGS. 2A-2D depict, in accordance with embodiments herein, FcγR stimulation in monocytes induces secretion of cytokines that favor the development of TH17 cells. Monocytes were stimulated with IC, LPS, Pam3, IFN-γ, Flagellin, or anti-CD40 Ab for the indicated time points. Secretion of various cytokines was analyzed by ELISA. FIGS. 2A-2D show a time-course of TL1A expression (FIG. 2A), IL-6 expression (FIG. 2B), IL-1β expression (FIG. 2C), and TGF-β1 expression (FIG. 2D).

FIGS. 3A-3G depict, in accordance with embodiments herein, TL1A enhances TH17 differentiation of naive CD4+ T cells. FIGS. 3A-3C: Naïve CD4+CD45RA+ human T cells were stimulated with plate-bound anti-CD3 and anti-CD28 antibodies with the addition of the indicated cytokines for 72 h. Culture supernatants were analyzed for IL-17 (FIG. 3A), IFNγ (FIG. 3B), IL-22 (FIG. 3C) after 72 h by ELISA. One representative experiment out of at least four independent experiments with similar results is shown. FIGS. 3D-3G: Naïve CD4+CD45RA+ human T cells were stimulated as described for 48 h and expression of RORC (FIG. 3D), RORA (FIG. 3E), IL-17A (FIG. 3F), and T-bet (FIG. 3G) was analyzed by real-time PCR. Data are representative of two independent experiments with similar results.

FIGS. 4A-4D depict, in accordance with embodiments herein, TL1A enhances IL-17, IFNγ, and IL-22 secretion from memory CD4+CD45RO+ T cells, and increases the number of IFN-γ- and IL-17-producing cells. (FIGS. 4A-4C) Human CD4+CD45RO+ T cells were stimulated with plate-bound anti-CD3 and anti-CD28 antibodies with the addition of the indicated cytokines. Culture supernatants were analyzed for IL-17 (FIG. 4A), IFNγ (FIG. 4B), and IL-22 (FIG. 4C) after 72 h by ELISA. Data represent the mean of duplicates±SD. One representative experiment out of at least four independent experiments with similar results is shown. FIG. 4D: Human CD4+CD45RO+ T cells were stimulated as described. After 72 h incubation cells were restimulated with PMA/Ionomycin for 5 h and the percentages of IFN-γ- and IL-17-producing cells were measured by intracellular cytokine staining using flow cytometry. Data are representative of four independent experiments with similar results.

FIGS. 5A-5C depict, in accordance with embodiments herein, TL1A enhances IL-17, IFNγ, and IL-22 secretion from committed CD4+CD45RO+CCR6+TH17 cells. FIGS. 5A-5C: Human CD4+CD45RO+CCR6+TH17 cells were stimulated with plate-bound anti-CD3 and anti-CD28 antibodies with the addition of the indicated cytokines. Supernatants were analyzed for IL-17 (FIG. 5A), IFNγ (FIG. 5B), and IL-22 (FIG. 5C) after 72 h by ELISA. Data represent the mean of duplicates±SD. One representative experiment out of three experiments with similar results is shown.

FIGS. 6A-6C depict, in accordance with embodiments herein, TL1A enhances IL-9 mRNA expression and secretion from committed CD4+CD45RO+CCR6+TH17 cells. Human CD4+CD45RO+CCR6+TH17 cells were stimulated with plate-bound anti-CD3 and anti-CD28 antibodies with the addition of the indicated cytokines. Fold increase of IL-9 mRNA was measured by real-time PCR (FIG. 6A). Supernatants were analyzed for IL-9 after 72 h by ELISA (FIG. 6B). Human CD4+CD45RO+CCR6+TH17 cells were stimulated with place bound anti-CD3 and anti-CD28 antibodies in the presence of the indicated cytokines for 24, 48, or 72 h. (FIG. 6C). Supernatants were analyzed for IL-9. Data represent the mean of duplicates±SD. One representative experiment out of three experiments with similar results is shown.

FIG. 7 depicts, in accordance with embodiments herein, TL1A enhances production of IL-9 and IL-22 producing cells. Human CD4+CD45RO+ T cells were stimulated with TL1A in the indicated concentrations. After 72 h incubation cells were restimulated with PMA/Ionomycin for 5 h and the percentages of IL-22 and IL-9 producing cells were measured by intracellular cytokine staining using flow cytometry. One representative experiment out of three experiments with similar results is shown.

FIG. 8 depicts, in accordance with embodiments herein, IL-9 receptor neutralizing antibodies inhibit TL1A-induced IL-22 secretion from committed CD4+CD45RO+CCR6+TH17 cells. Human CD4+CD45RO+CCR6+TH17 cells were stimulated with anti-CD3, anti-CD28, and TL1A in the presence of neutralizing IL-9 receptor antibodies or isotype controls for 2 days. Supernatants were analyzed for IL-22 by ELISA. Data represent the mean of duplicates±SD. One representative experiment out of three experiments with similar results is shown. **, p<0.01

FIG. 9 depicts, in accordance with embodiments herein, methods used by the inventors to determine if TL1A enhances the differentiation and/or activation of Th17 cells in humans.

FIG. 10 depicts, in accordance with embodiments herein, methods used by the inventors to determine if TL1A enhances the differentiation and/or activation of Th17 cells in humans.

FIG. 11 depicts, in accordance with embodiments herein, TL1A enhances the differentiation of Th17 cells from naïve CD45RA+ T cells. TL1A is able to substitute for IL-23 to achieve maximal induction of IL-17 production from naïve CD4+ T cells.

FIGS. 12A-12B depict, in accordance with embodiments herein, TL1A enhances the IFNgamma (FIG. 12A) and induces the IL-22 (FIG. 12B) production of Th17 cells.

FIG. 13A-C depicts, in accordance with embodiments herein, TL1A enhances induction of RORA (FIG. 13B) and T-bet mRNA (FIG. 13C) from CD45RA+ T cells, but not expression of RORC (FIG. 13A).

FIG. 14A-B depict, in accordance with embodiments herein, TL1A enhances the production of IL-17 (FIG. 14A) and IFNgamma (FIG. 14B) from memory CD45RO+ T cells.

FIG. 15 depicts, in accordance with embodiments herein, TL1A enhances the production of IL-17 from CD45RO+ T cells.

FIG. 16 depicts, in accordance with embodiments herein, TL1A minimally enhances IL-17 production by CD45RO+CCR6+Th17 cells. The data is consistent with the finding that TL1A has maximally enhancing effect under suboptimal stimulating conditions.

FIG. 17 depicts, in accordance with embodiments herein, TL1A greatly enhances IL-22 production by CD45RO+CCR6 Th17 cells. The inventors analyzed the expression of IL-22 in TH17 cells after stimulation with TL1A. TL1A alone greatly enhances the production of IL-22 in TH17 cells.

FIG. 18 depicts, in accordance with embodiments herein, a diagram noting that IL-9/Th9 is produced by several T cell subsets including Th17 cells.

FIG. 19 depicts, in accordance with embodiments herein, TL1A induces IL-9 mRNA.

FIG. 20 depicts, in accordance with embodiments herein, TL1A induces early IL-9 secretion in Th17 cells. First, the inventors measured IL-9 secretion upon stimulation with TL1A at different time-point. TL1A induces IL-9 secretion in TH17 cells early on (24 h), 11-fold increase over control cells. At later time-points (72 h) TGF-beta, IL-6, and TL1A have a synergistic effect on the secretion of IL-9, most likely through an indirect mechanism (up-regulation of IL-6/TGF-b receptor by TGF-b or IL-6 or TL1A). TL1A may induce early expression of IL-9 that in turn induces IL-22 expression.

FIG. 21 depicts, in accordance with embodiments herein, TL1A induces IL-9 production in a dose-dependent manner in Th17 cells. TL1A induces IL-9 in committed CD4+CD45RO+CCR6+TH17 cells. Human CD4+CD45RO+CCR6+TH17 cells were stimulated for 24 h with plate-bound anti-CD3 and anti-CD28 antibodies with the addition of TL1A at the indicated concentrations. Monensin was added for the last 4 h of stimulation and cells were stained for intracellular IL-9 and IL-22. Data are representative of 3 different experiments.

FIG. 22 depicts, in accordance with embodiments herein, TL1A induces IL-9 secretion in Th17 cells in a dose-dependent manner. In parallel, the inventors measured IL-9 in the supernatants of stimulated cells. The inventors observed a dose-dependent increase of IL-9 secretion after stimulation with increasing doses of TL1A at 24 and 48 h.

FIG. 23 depicts, in accordance with embodiments herein, IL-9R neutralizing Ab blocks TL1A-induced IL-22 secretion. The inventors used neutralizing anti-IL-9 receptor antibodies to block the TL1A-induced induction of IL-22. Neutralizing anti-IL-9R Ab blocks IL-22 in a dose-dependent manner. A dosage of 100 ug/ml results in a reduction of IL-22 secretion back to control levels. Left panel: secretion of IL-22 in response to TL1A-absolute values. Right panel: % of inhibition of TL1A-induced IL-22. 24 h time-point.

FIG. 24 depicts, in accordance with embodiments herein, a diagram of overall results.

FIGS. 25A-25B depict, in accordance with embodiments herein, sequences for probes and primers for the respective genetic loci. FIG. 25A depicts, in accordance with embodiments herein, sequences for probes and primers for the respective genetic loci. FIG. 25B depict, in accordance with embodiments herein, sequences for probes and primers for the respective genetic loci.

FIG. 26A-26F depict, expression analysis by the inventors of TL1A-induced signaling pathways (including 76 genes up-regulated by TL1A and 90 genes down-regulated by TL1A, comprising a signature of the TL1A signaling pathway). Together, the upregulation and downregulation patterns are part of an overall TL1A signaling profile. (FIG. 26A) depicts genes down regulated in response to TL1A. (FIG. 26B) depicts genes down regulated in response to TL1A. (FIG. 26C) depicts genes down regulated in response to TL1A. (FIG. 26D) depicts genes up regulated in response to TL1A. (FIG. 26E) depicts genes up regulated in response to TL1A. (FIG. 26F) depicts genes up regulated in response to TL1A.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Hornyak, et al., Introduction to Nanoscience and Nanotechnology, CRC Press (2008); Singleton et al., Dictionary of Microbiology and Molecular Biology 3rd ed., J. Wiley & Sons (New York, NY 2001); March, Advanced Organic Chemistry Reactions, Mechanisms and Structure 7th ed., J. Wiley & Sons (New York, NY 2013); and Sambrook and Russel, Molecular Cloning: A Laboratory Manual 4th ed., Cold Spring Harbor Laboratory Press (Cold Spring Harbor, NY 2012), provide one skilled in the art with a general guide to many of the terms used in the present application. One skilled in the art will recognize many methods and materials similar or equivalent to those described herein, which could be used in the practice of the present invention. Indeed, the present invention is in no way limited to the methods and materials described.

As disclosed herein, the inventors determined if TL1A enhances the differentiation and/or activation of TH17 cells in humans. TL1A is induced in Antigen-presenting cells in response to enteric bacteria and Fe gamma receptor signaling. Variation in TNFSF15, the TL1A gene, may contribute to Crohn's Disease and ulcerative colitis. Haplotypes composed of 5 TNFSF15 SNPs were observed to confer significant Crohn's Disease risk (haplotype A) and protection (haplotype B), using both case/control and family-based study designs. As further described herein, the inventors found that TL1A induces IL-22 and IL-17 from peripheral blood TH17 cells. Additionally, the inventors identified TL1A-induced IL-9 as a cytokine that induces IL⋅22 secretion from TH17 cells. Furthermore, the inventors demonstrate that blockage of IL-9 using neutralizing antibodies prevents TL1A-induced IL⋅22 secretion in TH17, suggesting a pathway for therapeutic intervention.

In one embodiment, the present invention provides a method of diagnosing a disease in a subject by obtaining a sample from the subject, subjecting the sample to an assay adapted to determine the presence or absence of TL1A expression and/or TL1A risk haplotype, and diagnosing the disease based on the presence of elevated TL1A expression and/or TL1A risk haplotype in the subject. In another embodiment, the subject is human. In another embodiment, the subject is Asian. In another embodiment, the subject has elevated Th17 signature cytokines. In another embodiment, the sample is taken from the peripheral blood and/or intestinal biopsy of the subject. In another embodiment, the subject is treated by administration of inhibitor of TL1A and/or administration of inhibitor of one or more Th17 signature cytokines. In another embodiment, the Th17 signature cytokines include IL-17, IL-22 and/or IL-9. In another embodiment, the disease is an inflammatory disease. In another embodiment, the disease is inflammatory bowel disease (IBD). In another embodiment, the disease is rheumatoid arthritis.

In one embodiment, the present invention provides a method of treating an inflammatory condition in a subject, comprising determining the presence of elevated TL1A expression, Th17 signature cytokines, and/or one or more TL1A genetic risk variants, and treating the inflammatory condition in the subject. In another embodiment, the subject is human. In another embodiment, the subject is Asian. In one embodiment, the inflammatory condition is inflammatory bowel disease. In another embodiment, the inflammatory condition is treated by administering a therapeutically effective dosage of inhibitor of TL1A and/or inhibitor of one or more Th17 signature cytokines. In another embodiment, the one or more Th17 signature cytokines include IL-17, IL-22 and/or IL-9. In another embodiment, the treatment is administered in conjunction with an anti-inflammatory therapeutic. In another embodiment, the treatment is administered by direct injection to the subject.

In one embodiment, the present invention provides a composition comprising a therapeutically effective dosage of inhibitor of TL1A and/or inhibitor of Th17 signature cytokines, and an acceptable carrier. In another embodiment, the inhibitor of TL1A is a TL1A antibody.

In one embodiment, the present invention provides a method of treating a disease in a subject by providing a composition comprising a therapeutically effective dosage of inhibitor of TL1A and/or inhibitor of Th17 signature cytokines, and administering the composition to the subject. In another embodiment, the disease is Inflammatory Bowel Disease (IBD). In another embodiment, the disease is arthritis.

As readily apparent to one of skill in the art, a variety of methods can be used to determine the presence or absence of a risk variant allele or haplotype, such as a TL1A (TNFSF15) genetic risk variant, and/or TL1A (TNFSF15) genetic risk haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

As further disclosed herein, the inventors demonstrate that FcγR signaling leads to concomitant induction of TL1A, IL-6, TGF-β, and IL-23, a cytokine milieu that fosters the development of TH17 cells. TL1A, in combination with TGF-β and IL-6, promoted differentiation of human TH17 cells from naive CD4+ T cells. Additionally, TL1A in combination with TGF-β and IL-6 enhanced IL-17 production from CD4+CD45RO+ memory T cells and induced IL-17/IFN-γ producing TH17 cells. In contrast, TL1A alone induced high levels of IL-22 in naïve and memory CD4+ T cells. TL1A also enhanced IL-17 and IL-22 production by committed CD45RO+CCR6+TH17 cells suggesting that TL1A is able to induce TH17 differentiation and enhances IL-17 secretion from committed TH17 cells. Thus, in one embodiment, TL1A provides a target for therapeutic intervention in chronic inflammatory diseases.

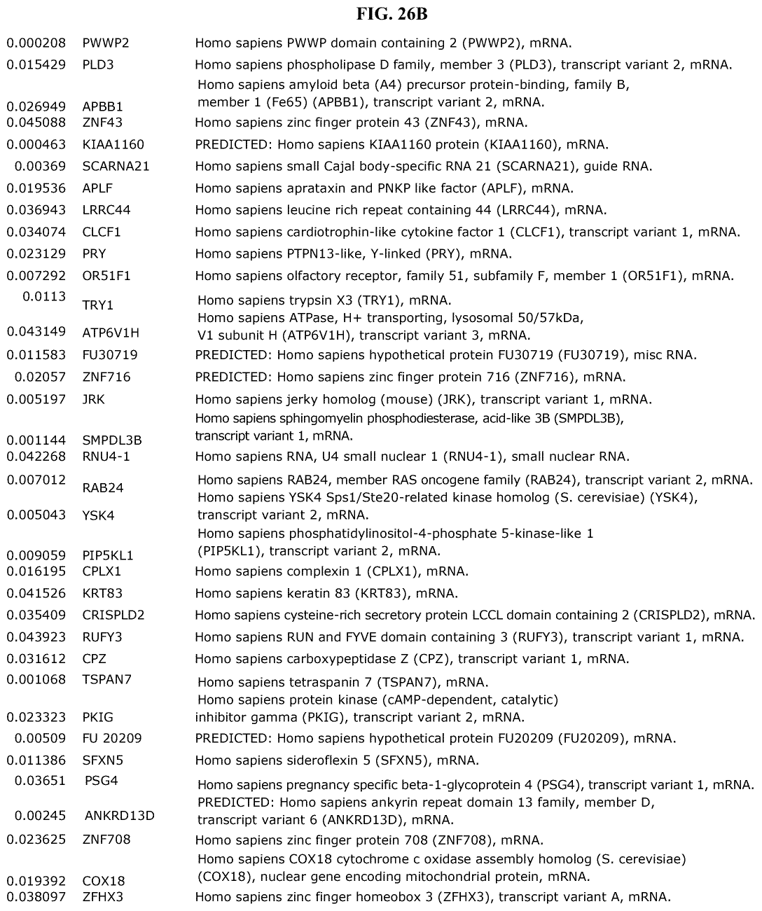

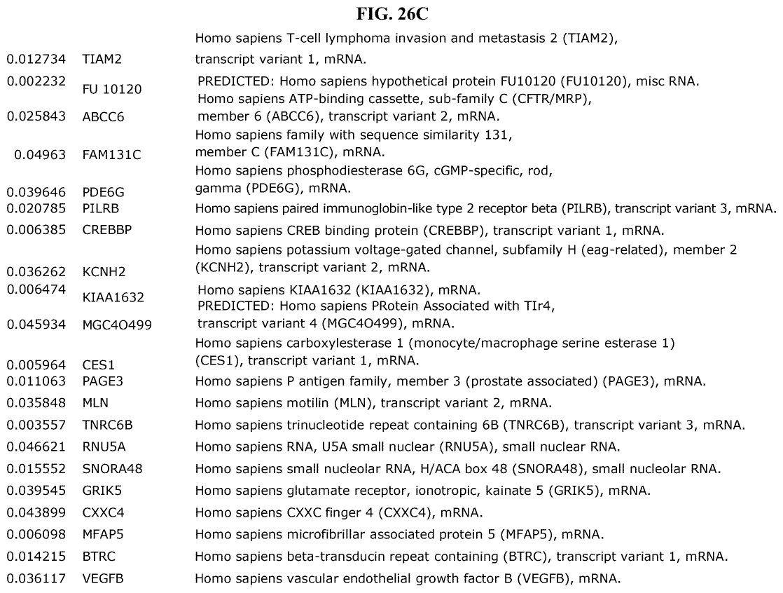

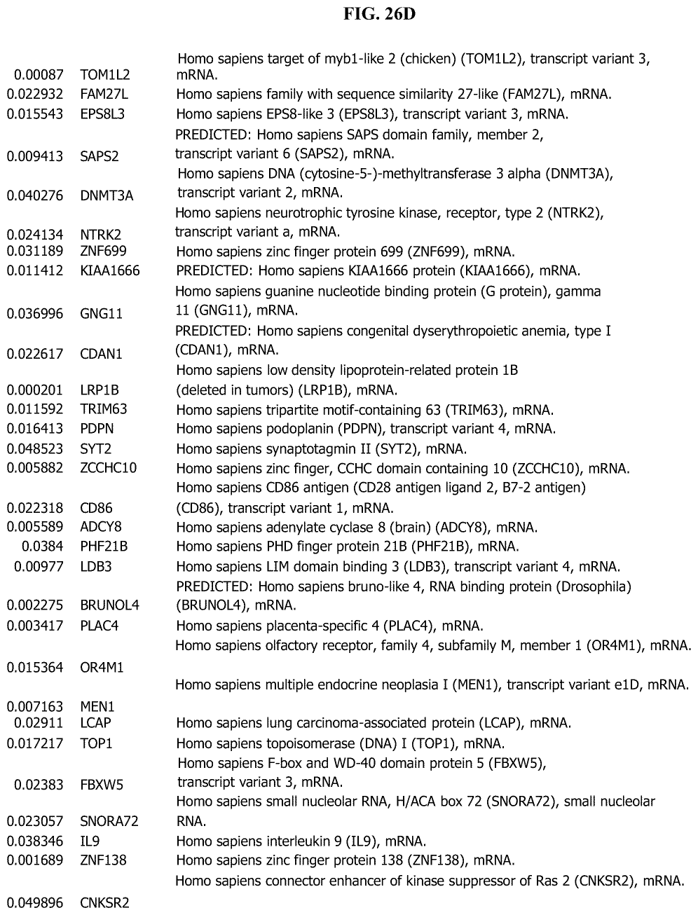

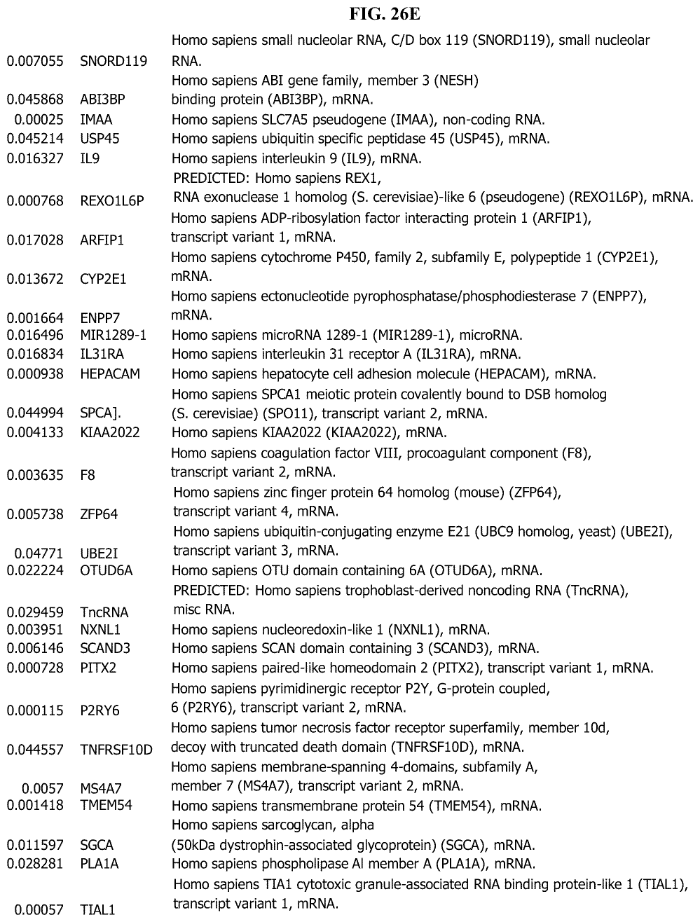

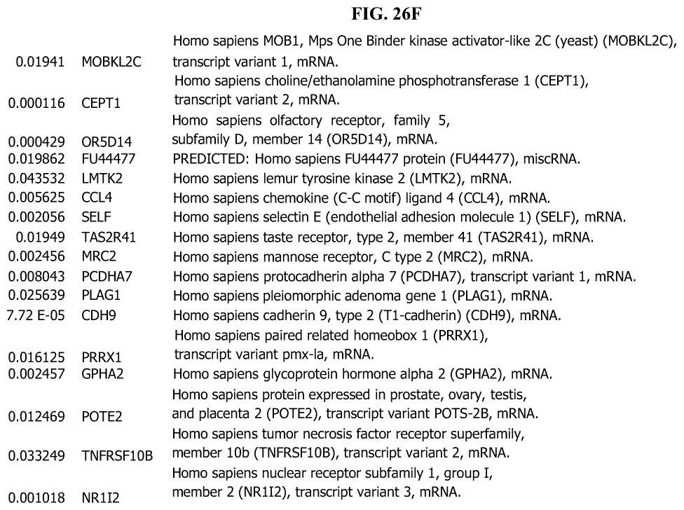

In one embodiment, the present invention provides a method of treating a disease, comprising obtaining a sample from a subject, assaying the sample to diagnose a disease based on the presence of TH17 cell differentiation, treating the subject by administering a therapeutically effective dosage of a composition comprising one or more inhibitors of TL1A. In another embodiment, the TH17 cell differentiation is from human naïve CD4+ T cells. The disease may also be in inflammatory related disease. In another embodiment, the disease is psoriasis, arthritis, experimental autoimmune encephalomyelitis, or colitis. In another embodiment, diagnosing the disease comprises determining the presence of a TL1A signaling profile. In another embodiment, the TL1A signaling profile comprises the presence of a downregulation of one or more genes listed in FIGS. 26A-26C herein. In another embodiment, the TL1A signaling profile comprises the presence of an upregulation of one or more genes listed in FIGS. 26D-26F herein. In another embodiment, the one or more inhibitors of TL1A is an inhibitor of the DR3 receptor. In another embodiment, the disease is intestinal inflammation.

In another embodiment, the present invention provides a method of treating an inflammatory disease in a subject, comprising determining the presence of a TL1A signaling profile in a subject, and treating the inflammatory disease by inhibiting TH17 differentiation from CD4+ T cells. In another embodiment, the TL1A signaling profile comprises the presence of a downregulation of one or more genes listed in FIGS. 26A-26C herein. In another embodiment, the TL1A signaling profile comprises the presence of an upregulation of one or more genes listed in FIGS. 26D-26F herein. In another embodiment, inhibiting TH17 differentiation comprises administering one or more inhibitors of TL1A. In another embodiment, inhibiting TH17 differentiation further comprises inhibiting IL-17 secretion from TH17 cells.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VIC™ to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,). Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al., (Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence, generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (AnalyzingDNA) New York, Cold Spring Harbor Laboratory Press (1997). In addition, one skilled in the art understands that multiple alleles can be detected in individual reactions or in a single reaction (a “multiplex” assay).

There are also many techniques readily available in the field for detecting the presence or absence of polypeptides or other biomarkers, such as determining the presence or absence of TL1A and/or one or more Th17 signature cytokines. For example, some of the detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

Similarly, there are any number of techniques that may be employed to isolate and/or fractionate biomarkers. For example, a biomarker may be captured using biospecific capture reagents, such as antibodies, aptamers or antibodies that recognize the biomarker and modified forms of it. This method could also result in the capture of protein interactors that are bound to the proteins or that are otherwise recognized by antibodies and that, themselves, can be biomarkers. The biospecific capture reagents may also be bound to a solid phase. Then, the captured proteins can be detected by SELDI mass spectrometry or by eluting the proteins from the capture reagent and detecting the eluted proteins by traditional MALDI or by SELDI. One example of SELDI is called “affinity capture mass spectrometry,” or “Surface-Enhanced Affinity Capture” or “SEAC,” which involves the use of probes that have a material on the probe surface that captures analytes through a non-covalent affinity interaction (adsorption) between the material and the analyte. Some examples of mass spectrometers are time-of-flight, magnetic sector, quadrupole filter, ion trap, ion cyclotron resonance, electrostatic sector analyzer and hybrids of these.

Alternatively, for example, the presence of biomarkers such as polypeptides maybe detected using traditional immunoassay techniques. Immunoassay requires biospecific capture reagents, such as antibodies, to capture the analytes. The assay may also be designed to specifically distinguish protein and modified forms of protein, which can be done by employing a sandwich assay in which one antibody captures more than one form and second, distinctly labeled antibodies, specifically bind, and provide distinct detection of, the various forms. Antibodies can be produced by immunizing animals with the biomolecules. Traditional immunoassays may also include sandwich immunoassays including ELISA or fluorescence-based immunoassays, as well as other enzyme immunoassays.

Prior to detection, biomarkers may also be fractionated to isolate them from other components in a solution or of blood that may interfere with detection. Fractionation may include platelet isolation from other blood components, sub-cellular fractionation of platelet components and/or fractionation of the desired biomarkers from other biomolecules found in platelets using techniques such as chromatography, affinity purification, 1D and 2D mapping, and other methodologies for purification known to those of skill in the art. In one embodiment, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

“Anti-TL1A therapy”, as used herein refers to therapeutic agents and methods that suppress TL1A gene expression, DR3 gene expression, or block the signaling of TL1A and DR3 (the receptor for TL1A) proteins. As used herein, the term “TL1A signaling inhibitor” (also interchangeably called as TL1A blocker or inhibitor, anti-TL1A reagent, agent, drug or therapeutic,) refers to any reagent that suppress responses to TL1A and/or inhibits the TL1A signaling, including inhibition of any molecular signaling step from the TL 1A ligand through its receptor to various downstream target molecules. A TL1A signaling inhibitor can be a small molecule; a nucleic acid such as siRNA, shRNA, and miRNA; a nucleic acid analogue such as PNA, pc-PNA, and LNA; an aptamer; a ribosome; a peptide; a protein; an avimer; an antibody, or variants and fragments thereof. Examples of the TL1A signaling inhibitor include but are not limited to an anti-TL1A antibody blocking TL1A-DR3 signaling, an anti-DR3 antibody blocking TL1A-DR3 signaling, a soluble decoy DR3 polypeptide (e. g., a soluble DR3-Fc fusion protein), or a nucleic acid antagonist of TL1A or DR3, such as an aptamer or antisense molecule targeting TL1A or DR3, or a combination thereof. In certain embodiments, the TL1A signaling inhibitor comprises an anti-TL1A antibody or a fragment thereof, an anti-DR3 antibody or a fragment thereof, a soluble decoy DR3 polypeptide, a nucleic acid antagonist of TL1A, or a nucleic acid antagonist of DR3, or a combination thereof.

Typical dosages of an effective amount of treatment or therapy can be in the ranges recommended by the manufacturer where known therapeutic molecules or compounds are used, and also as indicated to the skilled artisan by the in vitro responses in cells or in vivo responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, the therapy may be administered once a day (SID/QD), twice a day (BID), three times a day (TID), four times a day (QID), or more, so as to administer an effective amount of the therapy to the individual, where the effective amount is any one or more of the doses described herein.

In various embodiments, composition for treatment is administered at about 0.001-0.01, 0.01-0.1, 0.1-0.5, 0.5-5, 5-10, 10-20, 20-50, 50-100, 100-200, 200-300, 300-400, 400-500, 500-600, 600-700, 700-800, 800-900, or 900-1000 mg/kg, or a combination thereof. In various embodiments, the treatment or therapy is administered once, twice, three or more times. In some embodiments, the therapy is administered about 1-3 times per day, 1-7 times per week, or 1-9 times per month. Still in some embodiments, the treatment, or therapy is administered for about 1-10 days, 10-20 days, 20-30 days, 30-40 days, 40-50 days, 50-60 days, 60-70 days, 70-80 days, 80-90 days, 90-100 days, 1-6 months, 6-12 months, or 1-5 years. Here, “mg/kg” refers to mg per kg body weight of the individual. In certain embodiments, the therapy is administered to a human.

In accordance with the invention, the therapy may be administered using the appropriate modes of administration, for instance, the modes of administration recommended by the manufacturer. In accordance with the invention, various routes may be utilized to administer the IBD therapy of the claimed methods, including but not limited to aerosol, nasal, oral, transmucosal, transdermal, parenteral, enteral, topical, local, implantable pump, continuous infusion, capsules and/or injections. In various embodiments, the IBD therapy is administered topically, intravascularly, intravenously, intraarterially, intramuscularly, subcutaneously, intraperitoneally, intranasally, or orally.

The various methods and techniques described above provide a number of ways to carry out the invention. Of course, itis to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Conclusions

TL1A enhances differentiation of Th17 cells from naïve CD4+ T cells.
TL1A increases % of IL-17 and IL-17/IFNg double positive CD45RO+ T cells.
TL1A induces the expression of RORA and T-bet but has no effect on the expression of RORC.
TL1A induces IL-22 secretion in Th17 cells (CD45RO+CCR6+ T cells).
TL1A induces IL-9 secretion in Th17 cells.

### Example 2

### Diagnostics and Treatments

The inventors found that TL1A plays an important role in the induction of Th17 signature cytokines including IL-17, IL-22, and IL-9. Patients carrying a risk haplotype for the TL 1A gene have elevated expression of TL 1A. In one embodiment, the present invention provides a diagnostic where these patients will also have elevated TL1A-induced TH17 signature cytokines that could be measured as a diagnostic test. That diagnostic test would help to identify specific patient population that would benefit the most from inhibition of either TL1A or the TH17 signature cytokines. In another embodiment, the present invention provides a companion diagnostic test for patients with IBD or rheumatoid arthritis with ongoing inflammation to determine the degree of inflammation and/or identify potential treatment options. In another embodiment, patients with risk polymorphisms for TL1A would be identified as a subgroup that would benefit from treatment. Further, TL1A single nucleotide polymorphisms may be strongly associated with the development and severity of IBD particularly in the Asian population.

### Example 3

### Overall

TL1A, a member of the TNF superfamily, mediates a strong co-stimulation of TH1 cells by enhancing IFN-γ production by peripheral CD4+ and mucosal CCR9+ T cells. TL1A plays an important role in the development of chronic colitis, experimental autoimmune encephalomyelitis, and allergic lung inflammation by modulating TH1, TH17, and TH2 responses suggesting an important role in chronic inflammatory processes. We have shown that TL1A is induced in antigen-presenting cells in response to FcγR signaling. Here, we demonstrate that FcγR signaling leads to concomitant induction of TL1A, IL-6, TGF-β, and IL-23, a cytokine milieu that fosters the development of TH17 cells. TL1A, in combination with TGF-β and IL-6, promoted differentiation of human TH17 cells from naive CD4+ T cells. Additionally, TL1A in combination with TGF-β and IL-6 enhanced IL-17 production from CD4+CD45RO+ memory T cells and induced IL-17/IFN-γ producing TH17 cells. In contrast, TL1A alone induced high levels of IL-22 in naïve and memory CD4+ T cells. TL1A also enhanced IL-17 and IL-22 production by committed CD45RO+CCR6+TH17 cells suggesting that TL1A is able to induce TH17 differentiation and enhances IL-17 secretion from committed TH17 cells. Thus, in one embodiment, TL1A provides a target for therapeutic intervention in chronic inflammatory diseases.

### Example 4

### Methods and Materials

### Isolation of CD14+ Monocytes From Blood:

Blood was obtained from healthy volunteers after informed consent in accordance with procedures established by the Cedars-Sinai Institutional Review Board. Plate-bound, cross-linked human IgG (IC) was prepared. Monocytes were incubated with IC for the indicated time-points.

### Isolation of CD4+ T Cells From PBMC and Cell Sorting:

CD4+ T cells were isolated from PBMC by negative selection using the human T lymphocyte Enrichment Set (BD Bioscience). CD4+ T cells were stained with PE-conjugated anti-CD45RO Ab and FITC-conjugated anti-CD45RA Ab (Caltag Laboratories). CD45RA+ or CD45RO+ cells were collected using the MoFlo™ High Performance Cell Sorter (Dako Cytomation). In some experiments the CD4+ T cells were stained for CD45RO (FITC-conjugated) and CCR6 (PE-conjugated) and CD45RO+/CCR6+ cells were collected.

### T Cell Stimulation:

T cells were stimulated with immobilized anti-CD3 (5 ug/ml, BD Bioscience) and anti-CD28 (2 ug/ml, Bristol-Myers Squibb) in the presence of TGF-β1 (3 ng/ml, PeproTech), IL-6 (50 ng/ml PeproTech), IL-23 (20 ng/ml, R & D Systems), TL1A (100 ng/ml, Fitzgerald Industries International, Acton, MA), and neutralizing antibodies to IL-4 (2 ug/ml) and IFN-γ (3 ug/ml, Biolegend) for three days or as indicated. In selected experiments neutralizing antibodies to IL-9, IL-9 receptor (BioLegend), or isotype controls were added at the time of stimulation.

### Real-Time PCR Analyses

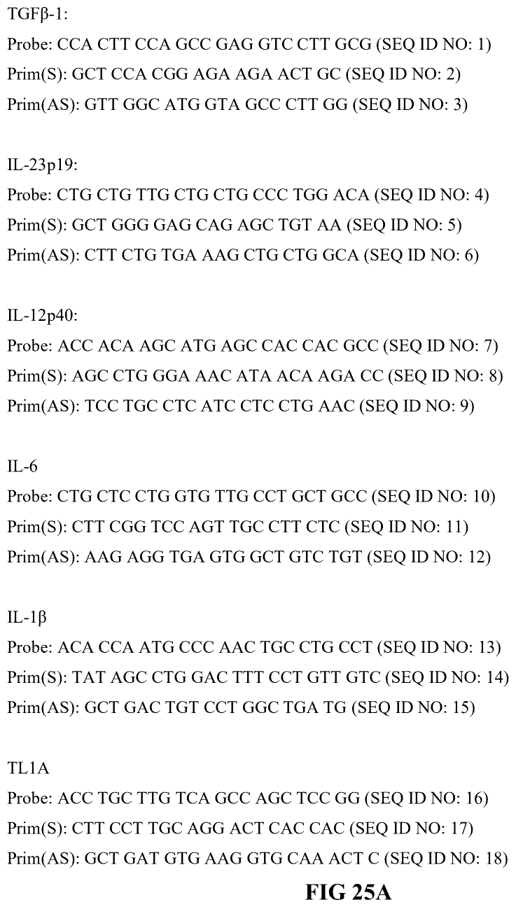

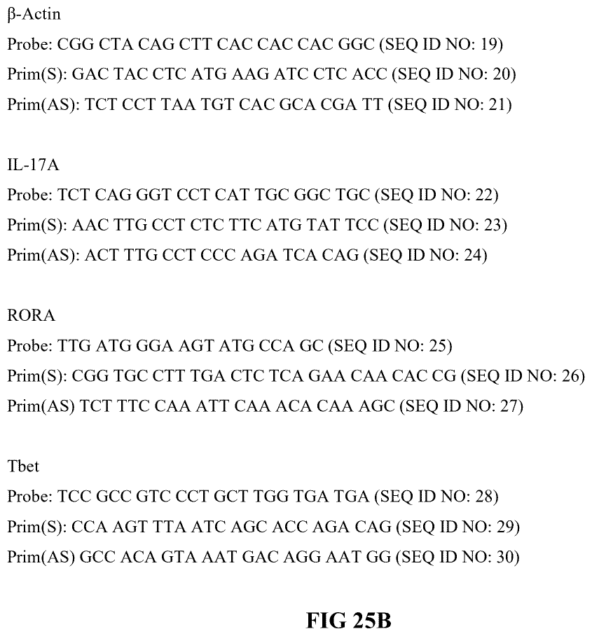

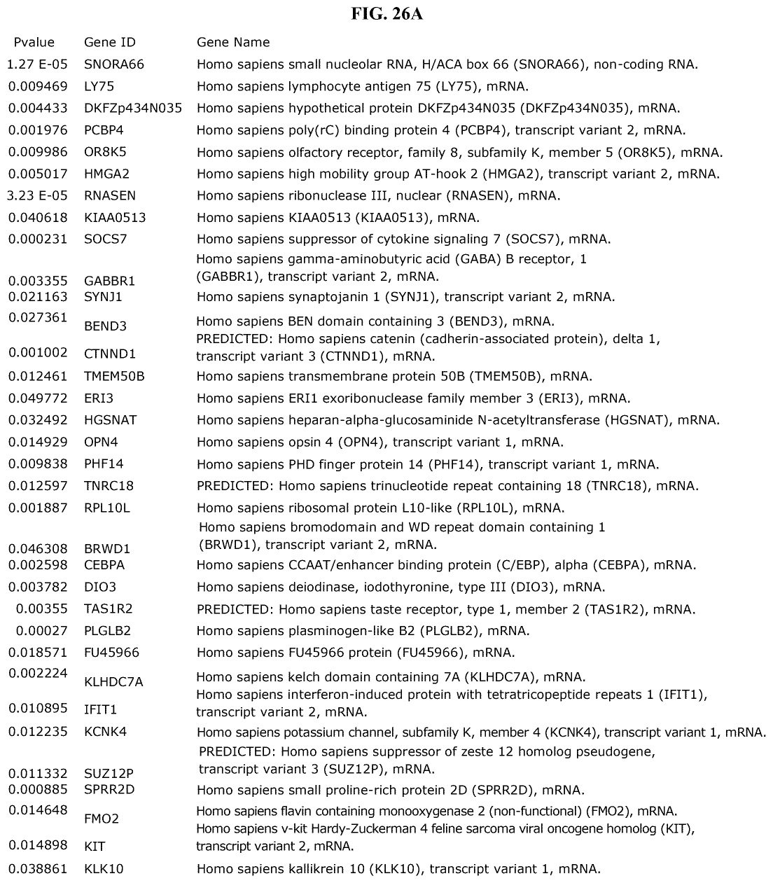

TL1A, TGFβ, IL-1β, IL-6, IL-23p19, IL-12p40, RORA, IL-17A (all Integrated DNA Technologies), and RORC (Applied Biosystems) transcripts were amplified by quantitative real-time RT-PCR. Primer/probe sequences described in FIGS. 25A-25B herein and as SEQ. ID NOS: 1-30.

### ELISA:

TL1A was quantified by ELISA. Human IL-17A, IFN-γ, TGF-β, IL-6, IL-1β, IL-9 (all eBioscience), IL-22 (R&D Systems) were quantified using ELISA kits.

### Flow Cytometry:

Cultured cells were stimulated with 50 nM of phorbol-12,13-dibutyrate and 1 ug/ml ionomycin for 2 h followed by Brefeldin A (Sigma-Aldrich, 10 ng/ml) stimulation for 4 h at 37 degrees C. Cells were harvested and stained for intracellular IL-17 (Alexa Fluor 647-IL-17), IFN-γ (PE-Cy 7-IFN-γ), IL-22 (PE-IL-22), IL-9 (eFluor 660-IL-9) or isotype controls utilizing the Fixation and Permeabilization kit (eBioscience). Cells were acquired on a CyAn™ ADP flow cytometer (Dako) and analyzed using FlowJo software (TreeStar Inc., Ashland, OR).

### Gene Expression Analysis:

RNA was isolated from stimulated T cells at the indicated time-points. cRNA amplification and biotin labeling were performed with 500 ng total RNA using Illumina TotalPrep RNA Amplification Kit (Ambion). cRNA yield was determined using a NanoDrop spectrophotometer (NanoDrop Technologies, Wilmington, DE), and 1.5 mg biotin-labeled cRNA was hybridized to HumanWG-6 v3 Expression Beadchips (Illumina). Data visualization and quality control were carried out with the Gene Expression module of GenomeStudio software (Illumina). Data were transformed by a variance stabilizing method and normalized by quantile normalization (implemented in the lumi package of R v2.11.1). Data were carried forward into analysis if the detection signal for all samples within at least one of the four groups (control-AA, CD-AA, control-GG, CD-GG) was positive (p value for detection<0.01). Thus 14 541 probes remained for analysis. The association of RNA expression was analyzed using a two-way analysis of variance (ANOVA) for this two-by-two factorial design across the four groups, as implemented in MultiExperiment Viewer (TM4 microarray software suite).

### Statistics:

Statistical significance was determined by Student's t test. A value of p<0.05 was considered to be statistically significant.

### Example 5

### Results

### Cytokine Expression Profile of FcγR Stimulated Human Monocytes:

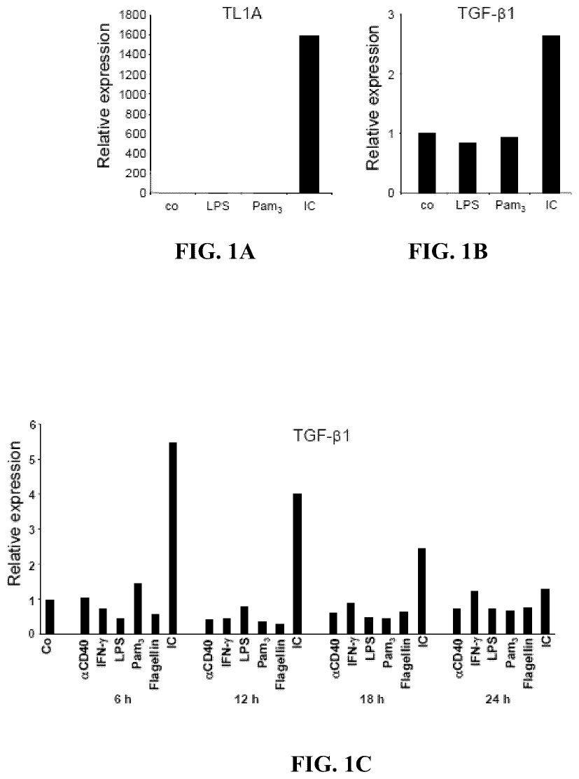

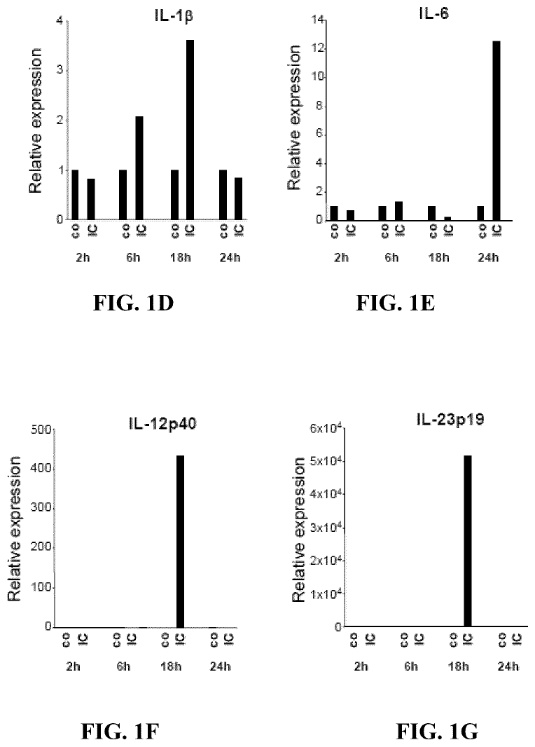

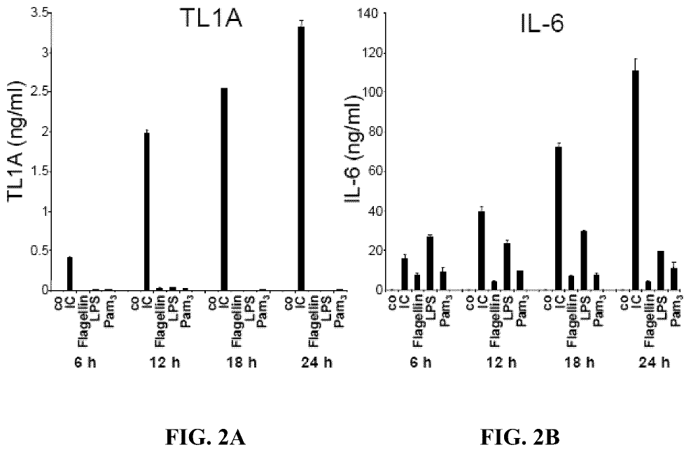

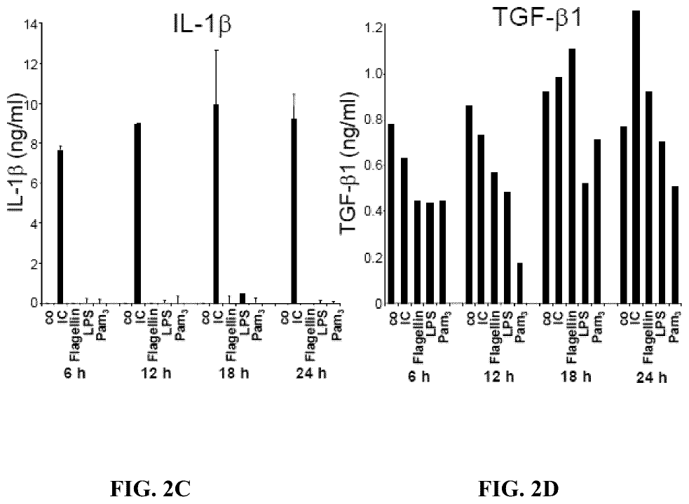

The inventors demonstrated that TL1A is a strong costimulator of TH1 responses. Furthermore, Fcγ Receptor signaling induces the expression of TL1A mRNA and subsequent secretion of TL1A protein in monocytes and dendritic cells (DC). However, to address whether TL1A also has an effect on the differentiation of the pro-inflammatory TH17 subset, the inventors first evaluated the expression pattern of cytokines that have been implicated in the differentiation of human TH17 cells in monocytes stimulated with immune complexes (IC), a strong inducer of TL1A in these cells. IC is a strong inducer of TL1A mRNA while TLR ligands (LPS as TLR4 ligand, Pam3 as TLR2 ligand) do not induce TL1A (FIG. 1A). Interestingly, only IC induced significant increases in TGF-β1 mRNA in monocytes while TLR ligands did not induce TGF-β1 (FIG. 1B). To evaluate if the lack of TGF-β1 induction by ligands other than IC is related to different kinetics in the induction of TGF-β1 the inventors performed extensive time-course experiments in monocytes using the TLR ligands LPS, Pam3, Flagellin, as well as IFN-γ, and anti-CD40 (FIG. 1C). TGF-β1 mRNA was only induced in IC stimulated monocytes while TLR ligands, IFN-γ, or anti-CD40 failed to induce TGF-β1 mRNA (FIG. 1C). Similar results were obtained from human monocyte-derived DC. The inventors evaluated the expression of cytokines that have been linked with the differentiation of human TH17 cells. IC induced IL-1β, IL-6, IL-12p40, and IL-23p19 mRNA (FIG. 1D-G). These real-time PCR data were confirmed by ELISAs. IC stimulation of monocytes resulted in significant secretion of IL-6, IL-1β, and TGF-β (FIG. 2B-2D). Interestingly, IC was the strongest inducer of IL-6 and IL-1β compared to several TLR ligands. Taken together, IC stimulation results in the concomitant secretion of TL1A and cytokines that have been implicated in the differentiation of TH17 cells (IL-6, IL-1β, IL-23, TGF-β).

### TL1A Enhances TH17 Differentiation of CD45RA+ Naive T Cells:

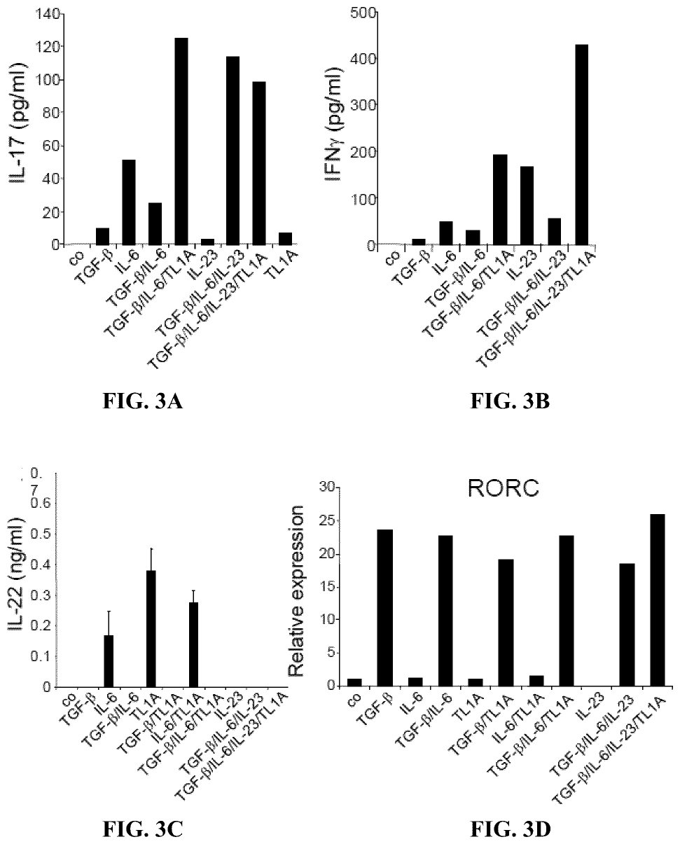

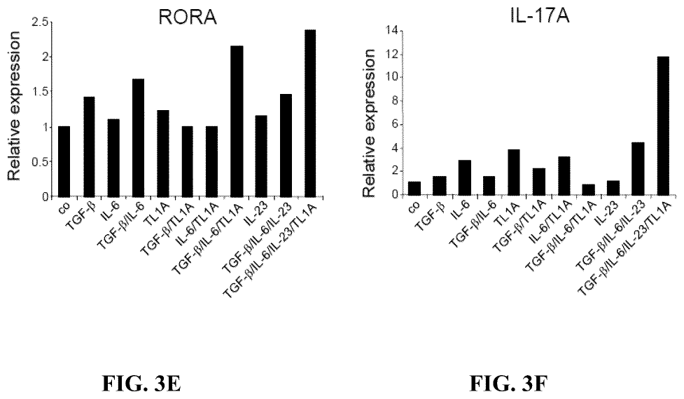

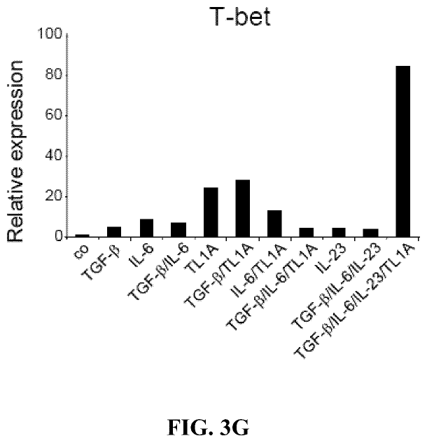

To establish a role of TL1A in the differentiation of human TH17 cells from naive CD45RA+ T cells, the inventors used high-speed flow cytometry for sorting CD45RA+ naive T cells from enriched CD4+ T cells isolated from PBMCs from healthy volunteers. The highly enriched naive CD4+CD45RA+ T cells were first differentiated with anti-CD3/CD28 in the presence of TGF-β+IL-6 in the presence or absence of TL1A. In comparison to cells treated with TGF-β+IL-6, cells treated with TGF-β+IL-6+TL1A secreted significantly more IL-17 (FIG. 3A). Interestingly, addition of IL-23 does not further enhance the secretion of IL-17. TL1A has been shown to strongly enhance IFN-γ production of CD4+ T cells (4). Next, the inventors analyzed the IFN-γ production of naive CD45RA+ T cells that have been stimulated under TH17 polarizing conditions. TL1A enhances the production of IFN-γ from cells stimulated with TGF-β+IL-6 (FIG. 3B). In contrast to the IL-17 secretion, TL1A and IL-23 have additive effects on the IFN-γ secretion when added to TGF-β+IL-6 (FIG. 3B). Interestingly, TL1A stimulation alone leads to the induction of IL-22 in naive CD45RA+ T cells (FIG. 3 C).

### TL1A Enhances RORA and T-bet Expression During Early TH17 Polarization:

To understand the molecular mechanisms by which TL1A enhances TH17 polarization, the inventors stimulated naive human CD4+CD45RA+ T cells under TH17 polarizing conditions in the absence or presence of TL1A and analyzed the expression of RORA and RORC mRNA (FIG. 3D, E). Consistent with previous findings TGF-β alone is sufficient to induce RORC mRNA and neither the addition of IL-6 nor TL1A enhances RORC expression (FIG. 3D). In contrast, TL1A enhances the expression of the transcription factor RORA in combination with TGF-β+IL-6+IL-23 (FIG. 3E). It has been previously reported that TH17 differentiation is regulated by both transcription factors, RORA and RORC to achieve maximal expression/differentiation of IL-17/TH17 cells. Enhancement of RORA expression was sufficient to lead to enhanced IL-17A mRNA expression by TL1A (FIG. 3F). Interestingly, stimulation with TL1A in combination with TGF-β+IL-6+IL-23 also led to a maximal induction of T-bet mRNA (FIG. 3G) confirming data of maximal induction of IFN-γ in TH17 cells polarized in the presence of TL1A.

### TL1A Enhances IL-17 Production by CD45RO+ Memory T Cells:

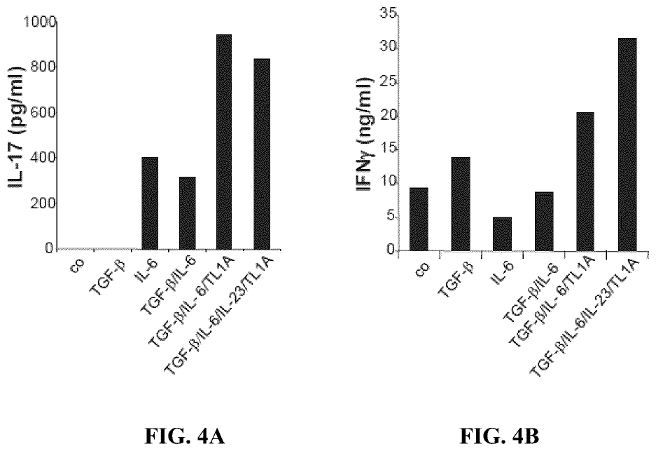

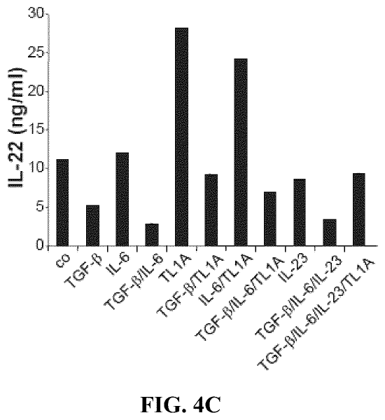

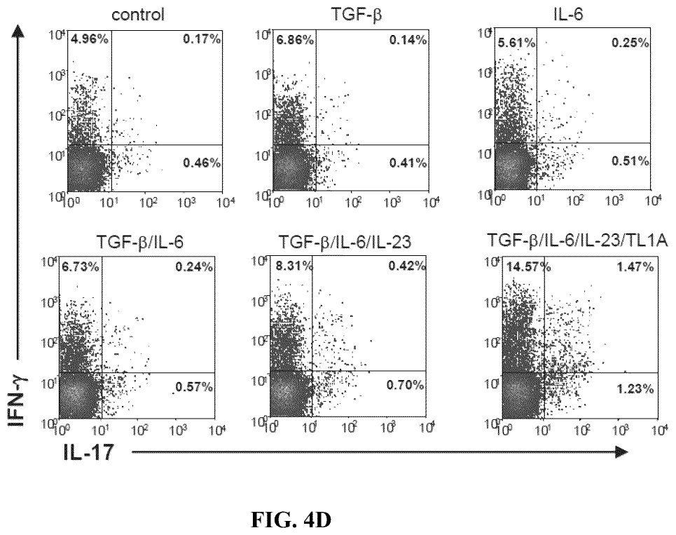

To further delineate the role of TL1A on TH17 development, the inventors analyzed if TL1A also has an effect on the IL-17 production by CD4+CD45RO+ memory T cells. Interestingly, similar to findings in naive T cells TL1A enhances the production of IL-17 in combination with TGF-β+IL-6 (FIG. 4A). Interestingly, addition of IL-23 does not further enhance the secretion of IL-17 from CD4+CD45RO+ memory T cells. In contrast, IL-23 and TL1A have additive effects on the IFN-γ secretion when added to TGF-β+IL-6 (FIG. 4B). When analyzed the secretion of IL-22, it was observed that TL1A alone leads to maximal secretion of IL-22 in CD4+CD45RO+ memory T cells (FIG. 4C). In contrast, TGF-β inhibits IL-22 secretion by TH17 cells and TL1A was not able to overcome the suppression by TGF-β. Furthermore, intracellular cytokine staining showed that TL1A in combination with TGF-β+IL-6+IL-23 enhances the percentage of IL-17+ cells (FIG. 4D). TL1A also enhances the IFN-γ+ cell population and induces a cell population of IL-17/IFN-γ double positive cells (FIG. 4D). These data confirm data demonstrating concomitant secretion of IL-17 and IFN-γ under TH17 polarizing conditions and simultaneous induction of RORA and t-bet by TL1A.

### TL1A Increases IL-17 and IL-22 Production in Committed TH17 Cells:

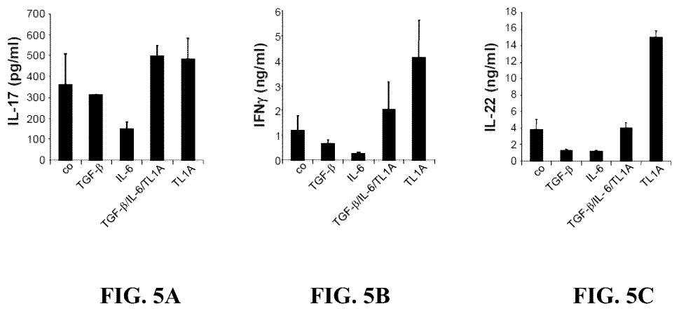

To determine if TL1A could enhance the production of IL-17 from committed TH17 cells, the inventors isolated human CD45RO+CCR6+ cells from peripheral blood and stimulated these cells with TL1A for three days. Stimulation of TH17 cells with TL1A enhances the secretion of IL-17 compared to unstimulated cells by approximately 30% (FIG. 5A). In contrast, treatment with TGF-β or IL-6 decreases the production of IL-17 in committed TH17 cells (FIG. 5A). The inventors also observed an enhancement of IFN-γ and IL-22 production by TL1A alone in committed TH17 cells (FIG. 5B, C).

### Increased IL-22 Production in Committed TH17 Cells by TL1A Stimulation is Driven by IL-9 Secretion:

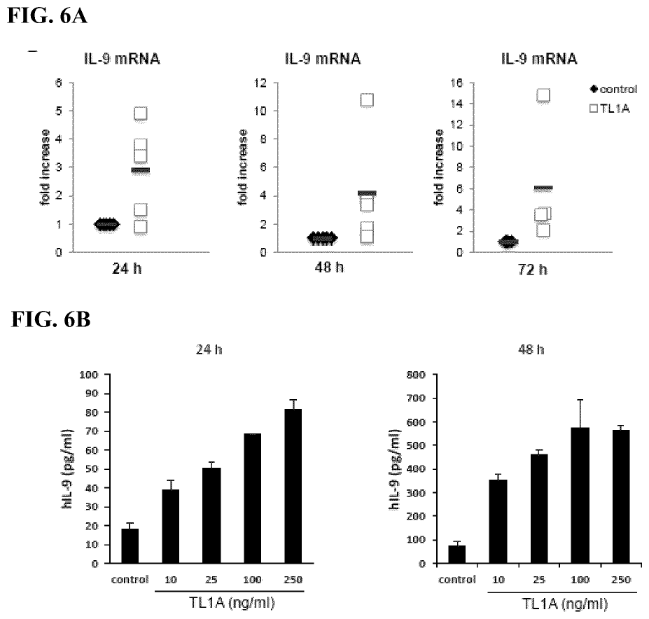

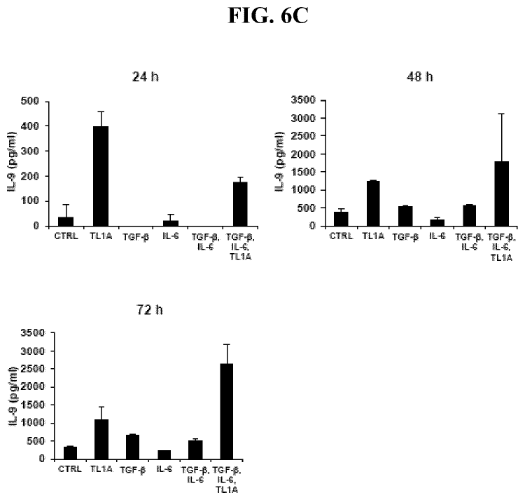

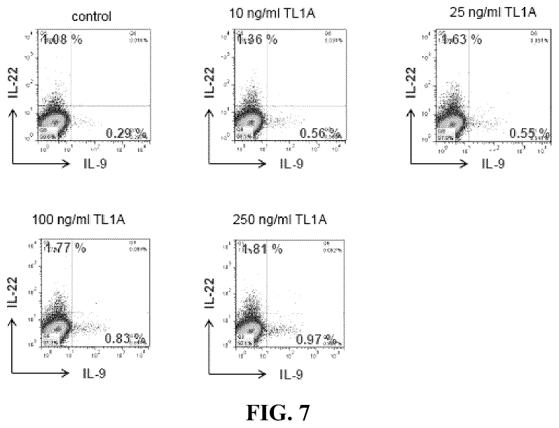

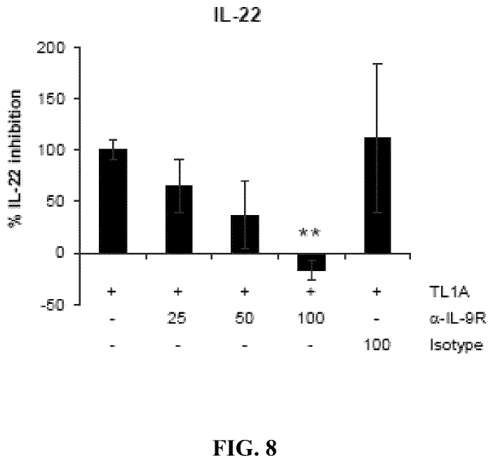

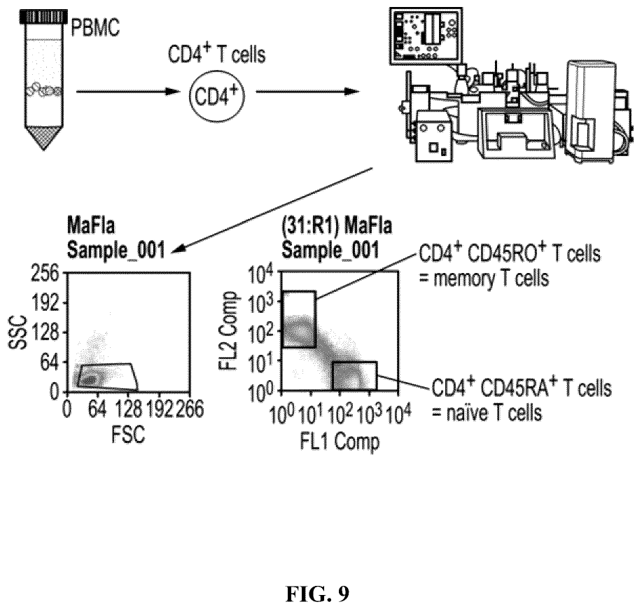

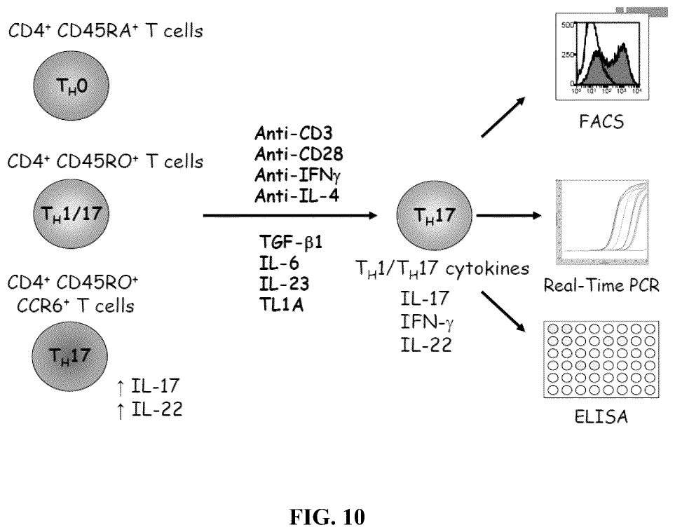

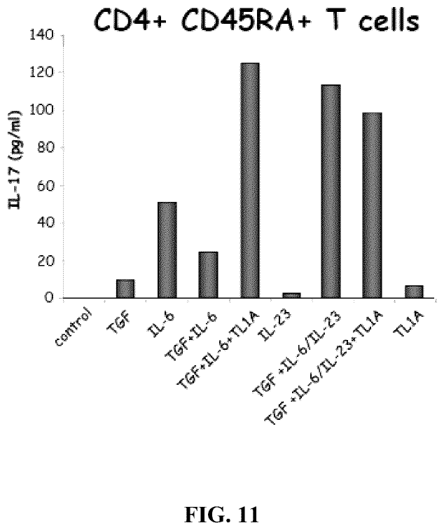

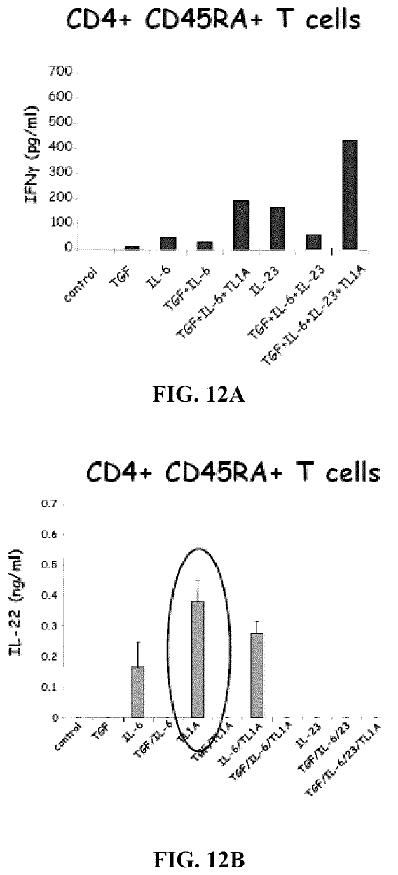

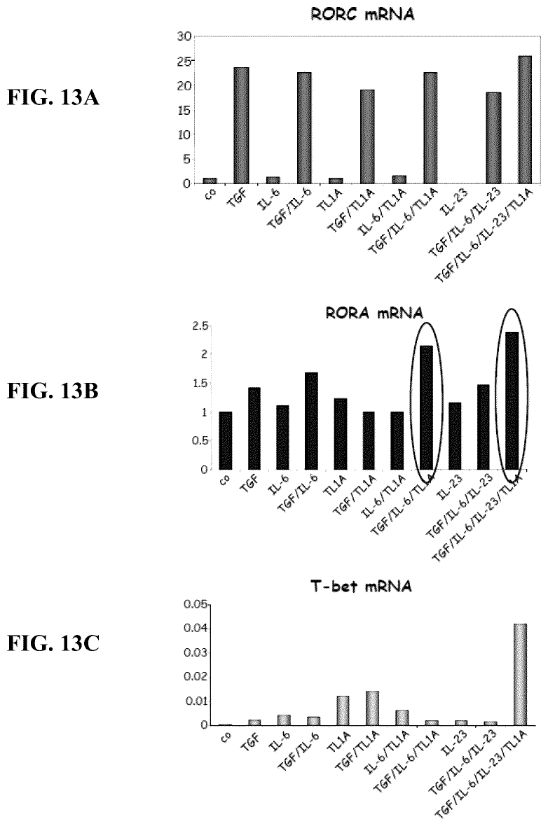

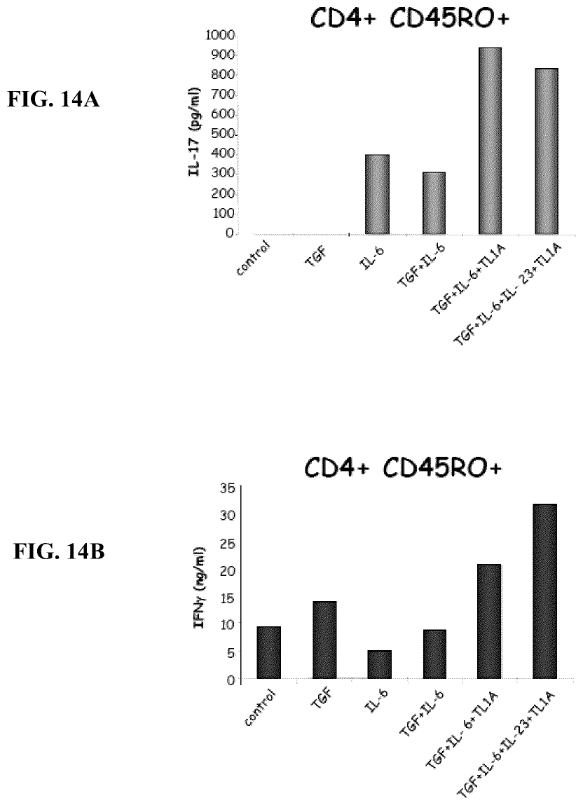

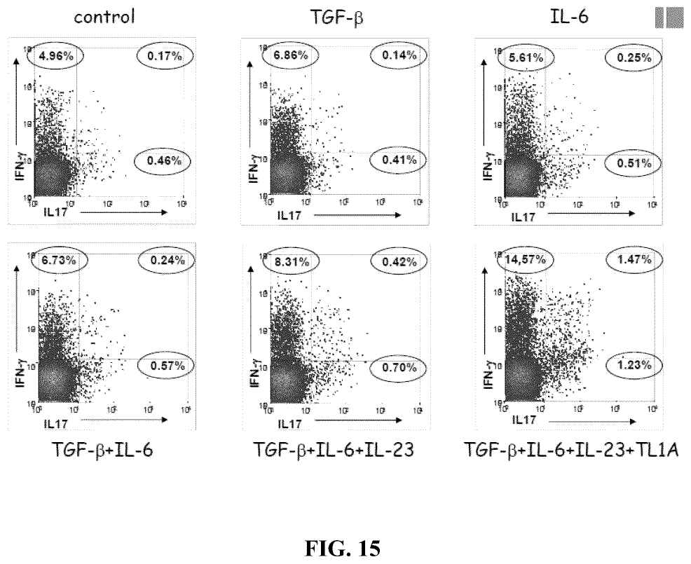

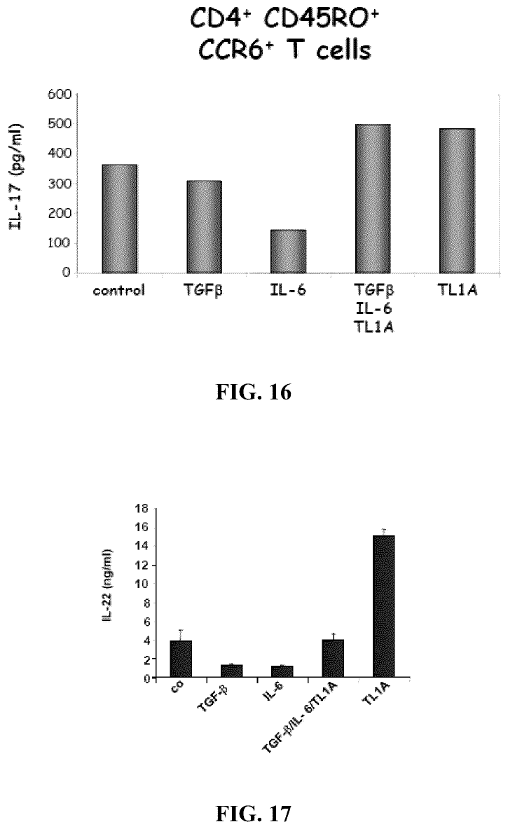

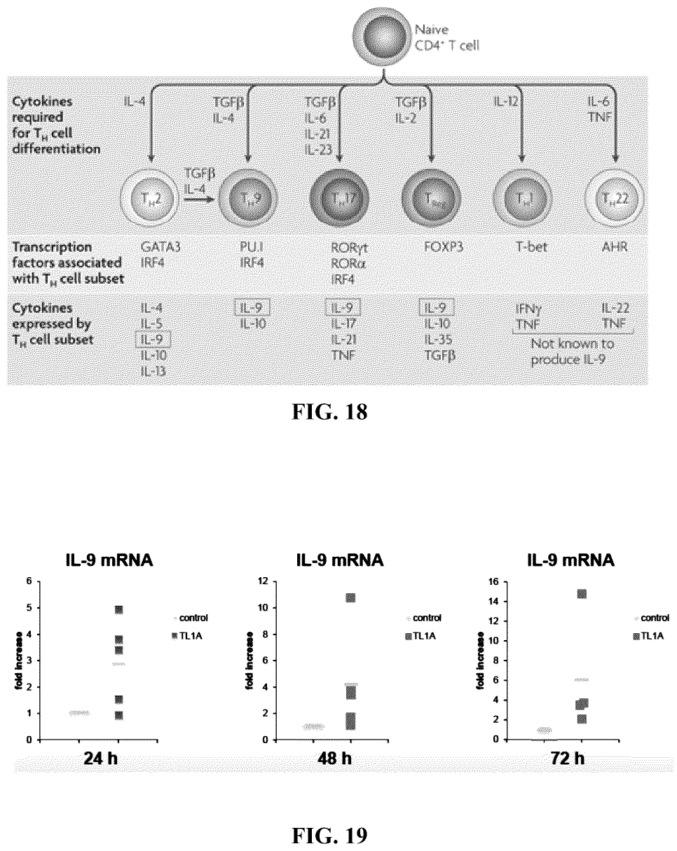

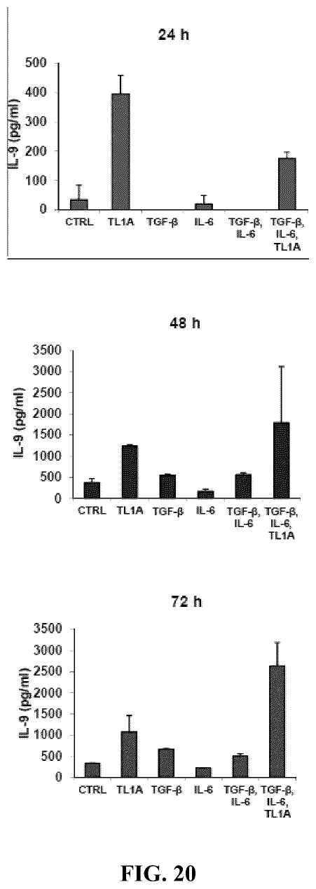

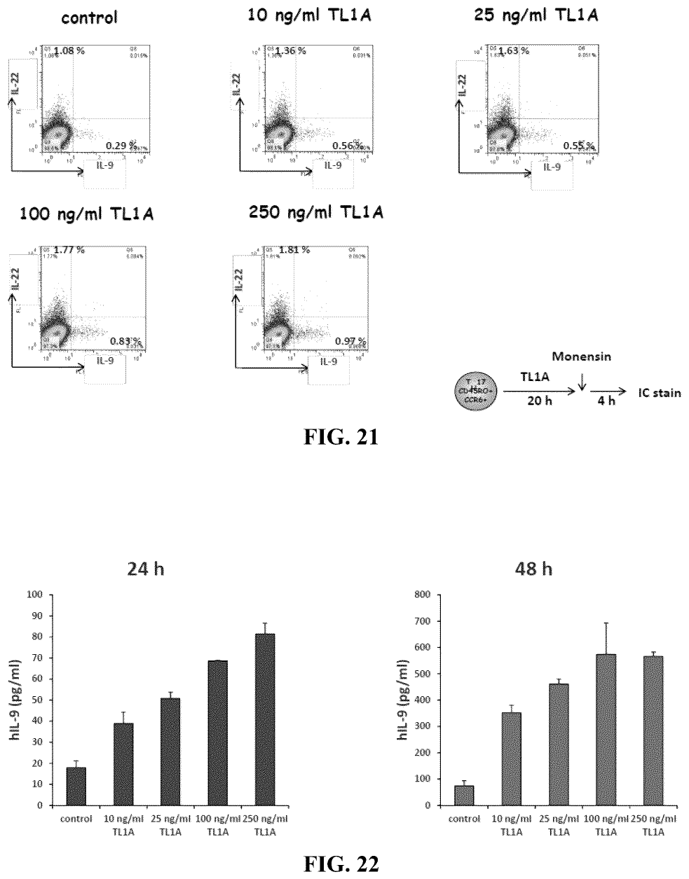

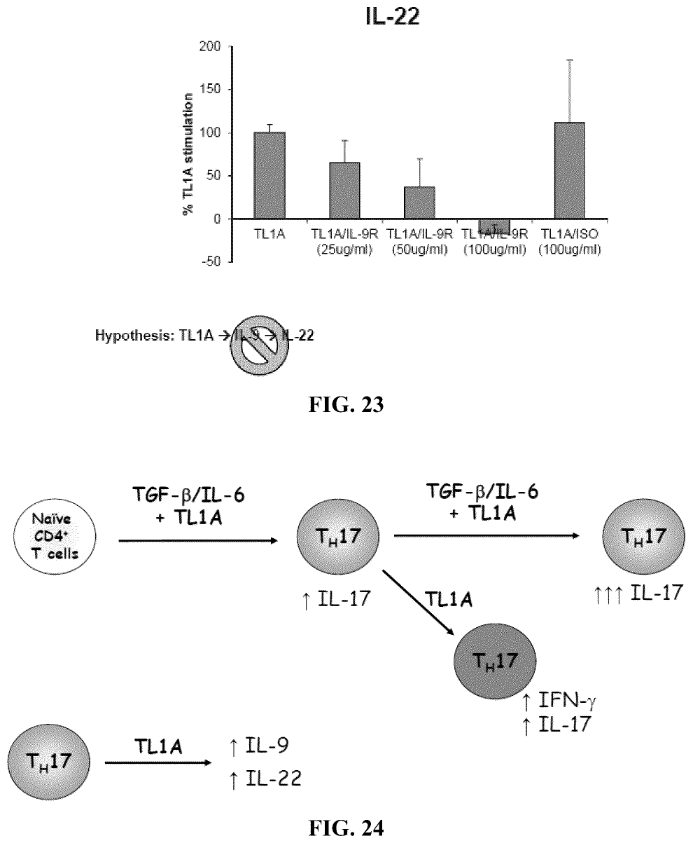

In order to determine the mechanism of TL1A-induced IL-22 production in TH17 cells the inventors performed global gene expression analysis of TL1A-stimulated TH17 cells (FIG. 6A). Stimulation of TH17 cells with TL1A resulted in the up-regulation of 76 genes by at least 2-fold and the down-regulation of 90 genes by at least 2-fold. The inventors observed that the expression of the cytokine IL-9 was up-regulated by TL1A stimulation. Since IL-9 has been described to be an inducer of IL-22 the inventors believed that the induction of IL-9 by TL1A could lead to the up-regulation of IL-22. First, they confirmed gene array data by quantitative real-time PCR. IL-9 mRNA expression was induced early on in TL1A-stimulated TH17 cells and remained up-regulated for up to 72 h (FIG. 6B). Next, confirmed TL1A induced IL-9 secretion in TH17 cells. They observed a dose-dependent increase of IL-9 secretion after stimulation with TL1A at 24 and 48 h (FIG. 6C). Concomitant with a dose-dependent increase of IL-9 by TL1A stimulation they also observed an increase in IL-22+ and IL-9+IL-22+ cells as detected by intracellular cytokine staining (FIG. 7). To prove the hypothesis that the TL1A-induced secretion of IL-22 is dependent on IL-9 they utilized anti-IL-9R neutralizing antibodies. According to the hypothesis they observed a dose-dependent inhibition of TL1A-induced IL-22 (FIG. 8). They observed similar results with neutralizing anti-IL-9 antibodies. These data demonstrate that TL1A induces IL-22 in TH17 cells via the early induction of IL-9.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, itis the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

1-10. (canceled)

11. A method of treating an inflammatory disease in a subject, comprising: obtaining a biological sample from the subject;
assaying the biological sample to diagnose the inflammatory disease based on the presence of TH17 cell differentiation; and
treating the subject by administering a therapeutically effective dosage of a composition comprising one or more inhibitors of tumor necrosis factor-like cytokine 1A (TL1A).

12. The method of claim 11, wherein the subject is human.

13. The method of claim 11, wherein the biological sample is taken from the peripheral blood and/or intestinal biopsy of the subject.

14. The method of claim 11, wherein the inflammatory disease is inflammatory bowel disease (IBD).

15. The method of claim 11, wherein the inflammatory disease is rheumatoid arthritis.

16. The method of claim 11, further comprising determining an expression level of one or more cytokines in the biological sample from the subject.

17. The method of claim 16, wherein the one or more cytokines comprises IL-17, IL-9 and/or IL-22.

18. The method of claim 11, further comprising determining an elevated presence of the TH17 cell differentiation in the biological sample relative to a control level of TH17 cell differentiation in an individual that does not have the inflammatory disease.

19. The method of claim 11, wherein the one or more inhibitors of TL1A is an anti-TL1A antibody.

20. The method of claim 11, wherein the one or more inhibitors of TL1A is an inhibitor of TH17 cytokines.

21. The method of claim 11, wherein treating the subject further comprises inhibiting IL-17 secretion of TH17 cells.

22. The method of claim 11, wherein the composition is administered in combination with an anti-inflammatory therapeutic.

23. The method of claim 11, wherein the administering of the composition is performed by direct injection to the subject.

24. The method of claim 14, wherein the IBD is ulcerative colitis (UC).

25. The method of claim 14, wherein the IBD is Crohn's disease (CD).

26. The method of claim 11, wherein the inflammatory disease is psoriasis, arthritis, or experimental autoimmune encephalomyelitis (EAE).

27. The method of claim 11, further comprising determining an elevated level of interleukin 17 (IL-17) in the biological sample relative to a control level of IL-17 in an individual that does not have the inflammatory disease.

28. The method of claim 11, further comprising determining a decreased presence of TH17 cell differentiation following the administering to the subject the one or more inhibitors of TL1A, wherein the decreased presence of TH17 cell differentiation is relative to the level of TH17 cell differentiation determined prior to the administering to the subject the one or more inhibitors of TL1A.
